(19) United States
(12) Patent Application Publication
(10) Pub. No.: US 2011/0202063 A1
(43) Pub. Date: Aug. 18, 2011

(54) APPARATUS FOR DELIVERY OF VISCOUS MATERIAL DURING SURGERY

(76) Inventors: Freddy Bonnin, Toulouse (FR); Alain Leonard, Caixon (FR)

(21) Appl. No.: 12/705,648

(22) Filed: Feb. 15, 2010

(51) Int. Cl. A61B 17/56

(52) U.S. Cl. 606/94

(57) ABSTRACT

Embodiments of the present disclosure are directed to devices, systems and methods for delivering viscous materials into a body, and more particularly, to devices, systems and methods for delivering bone fill material, biomaterials, and/or other flowable compounds into vertebrae, during, for example, a vertebroplasty procedure.

### BACKGROUND OF THE DISCLOSURE

1. Field of the Invention

This application describes a medical device or an apparatus for injecting bone fill material into a vertebrate body.

2. Background

Bone fill materials have been used in joint surgery as well as in vertebroplasty. The cement which serves as a junction between the bone and the implant must meet a certain number of requirements, in particular mechanical, but must also be non-toxic and biocompatible. Certain cements have even been studied for their bioactive properties, i.e. for their action which assists the adhesion and the cellular growth on the implant.

The composition of bone fill materials may vary, but essentially comprises of two-component materials: a powder component and a liquid component. The powder component may be composed of homopolymers of polymer of polymethyl methacrylate (PMMA) or PMMA and MMA co-polymer beads, accompanied by a radio-opacifer and calcium phosphate.

Polymeric cements become more viscous as the polymer chain grows by reacting directly with the double bond of a monomer. Polymerization begins by the “addition mechanism” in which a monomer becomes unstable by reacting with an initiator, a volatile molecule that is most commonly a radical (molecules that contain a single unpaired electron). Radicals bond with monomers, forming monomer radicals that can attack the double bond of the next monomer to propagate the polymer chain. Because radicals are so transient, initiators are often added in the form of an unreactive peroxide form that is stable in solution. Radicals are formed when heat or light cleaves the peroxide molecule. For applications in which high temperatures are not practical (such as the use of bone fill material in vivo), peroxide is cleaved by adding a chemical activator such as N,N-dimethyl-p-toludine. (Nussbaum DA et al., “The Chemistry of Acrylic Bone fill material and Implication for Clinical Use in Image-guided Therapy,” J. Vasc. Interv. Radiol. (2004); 15:121-126; the content of which is fully incorporated herein by reference).

Viscous cement is advantageous not only in reducing the risk of leakage, but also, because of its ability to infiltrate into the intravertebral cancellous bone (interdigitation). In addition, viscous material may reduce risk of fracture. Examples of commercially available viscous bone fill materials include, but are not limited to CMW® Nos. 1, 2 and 3 (DePuy Orthopaedics Inc., Warsaw, Ind., USA) and Simplex®-P and -RO (Stryker Orthopaedics; Mahwah, N.J., USA). These cements are characterized by a liquid phase after mixing and prior to achieving a viscosity of 500 Pascal second. In a typical use scenario, these previously available cements are poured, while in a liquid phase, into a delivery device.

Viscous bone fill material has an enhanced viscosity window before it sets. The viscosity, while even high, does not vary to a degree that influences injection parameters. The cement is generally sufficiently viscous to move fractured bone, such as vertebral plates of a collapsed vertebra, as it is injected. Injection of viscous cement contributes to fracture reduction and/or restoration of vertebral height.

Many patients can be effectively treated by the percutaneous injection of bone fill material into the fractured vertebral body. This procedure results in significant reduction in pain in approximately 80% of patients treated by vertebroplasty, in addition to strengthening of the diseased bone.

Vertebroplasty is a minimally invasive surgical technique that has been introduced to medically manage vertebral compression fractures. In this procedure, bone fill material is injected percutaneously, through a long cannula and into the vertebral cancellous bone. Once hardened, the cement reinforces mechanically the weakened vertebra. The principal benefit of this procedure is that up to 90% of the patients experience pain relief within 24 hours. (Jensen, M. E. et al., (1997), Am. J. Neuroradiol. 18: 1897-1904). The procedure aims to augment the weakened vertebral body and stabilize it. A dough of an injectable bone fill material is carefully injected directly into the fractured vertebral body.

Bone-cement delivery devices have been described. However, none are designed to allow controlled delivery by remotely controlling the bone fill material delivery process. Remote control delivery offers the advantage in that the person delivering is not exposed to any radiation or other potential exposures in an operating chamber. Accordingly, apparatus and methods for delivering bone fill material in an automated fashion into vertebrate body would be useful.

### SUMMARY OF THE DISCLOSURE

Embodiments of the present disclosure are directed to devices, systems and methods for delivering viscous materials into a body, and more particularly, to devices, systems and methods for delivering flowable materials into vertebrae, during, for example, a vertebroplasty procedure.

According to some embodiments, a bone fill material injection system is provided for injecting bone fill material into an area of bone. In one embodiment, the system comprises a bone fill material injection system for injecting bone fill material, by remote control, into an area of bone. This system comprises an injector, wherein the injector comprises a force applicator component, with a proximal end and a distal end, comprising a main body, a piston slidably disposed within the main body, and a force applicator (e.g., motor); a delivery reservoir component comprising a reservoir and an outlet. The force applicator component may be controlled by a remote control device. The motor may be a reduction motor. The delivery reservoir component may be releasably connected to the force applicator component. Further, a gasket may be placed between the delivery reservoir and the force applicator component.

In some embodiments, the bone fill material injection system comprises a piston that moves along a central axis within the main body of the injector. Optionally, the movement of the piston is powered and/or controlled by the motor placed at or near the proximal end of the force applicator component, and wherein the motor is connected to the piston. Preferably, the motor is connected to the piston with circlips. Preferably, the motor generates a force which is transferred to the piston for ejecting the bone fill material through a delivery outlet into an implant area.

In some embodiments of the bone fill material injection system, the motor includes an electric motor. In another embodiment, the injector injects the bone fill material where at least one of the volume and flow rate of injected cement is accurately controlled via the motor. In another embodiment, the remote control device controls the motor via a wire or wireless connection.

In some embodiments, the remote control comprises one or more (e.g., 2, 3, 4, 5, 6 or more) of: a first screen for showing the time spent and remaining for injecting bone fill material; a second screen for showing the cumulative volume of the injected bone fill material; a button for injecting low volume of bone fill material; a button for injecting moderate volume of bone fill material; a button for injecting high volume of bone fill material; a button of departure countdown; a switch button to turn on or off the remote control; a light to indicate that transfer has begun; a light to indicate that injection has begun; a light to indicate that injection has ended; and a button to stop injection in an emergency.

In an alternative specific embodiment of the bone fill material injection system, the delivery outlet is connected to a cannula or a syringe needle. In another embodiment, the shape of the force applicator component is cylindrical.

In an alternative specific embodiment of the bone fill material injection system, the system further comprises a mixer. The mixer may comprise two or more of a mixing reservoir; a rotating mixer; a crank; and a cover. The crank may be connected at one end to the handle and at the other end to the rotating mixer, and the rotating mixer is rotated with the crank for mixing a powder component and a liquid component of the bone fill material in the mixing reservoir. In some embodiments, the mixer further comprises a scraper, wherein the scraper moves along the body of the mixer by rotating along matched threads within the reservoir and is separable from the reservoir by rotating in the reverse direction, and holds down the bone fill material in the reservoir once the cover is removed. In some embodiments, the rotating mixer is in a shape and form (e.g., oval shape) that creates a shearing effect to mix the powder and liquid components to a homogenous bone fill material mix.

In an alternative specific embodiment of the bone fill material injection system, the system further comprises an extractor used for manual transfer of bone fill material from the mixer to the drug reservoir of the injector; wherein the extractor comprises a transfer piston, which is assembled to the cement mixing reservoir at distal end and to the force applicator of the injector at proximal end. In some embodiments, pressure on the upper edge of the transfer piston causes the piston to descend into the mixing reservoir, thereby displacing the cement upwards into the main body of the force applicator.

According to some embodiments, there is provided a method for injecting bone fill material into an implant area comprising: mixing a powder component and a liquid component of bone fill material together in a mixer, using an extractor to transfer the mixed bone fill material from the mixer to a motor powered injector; and injecting the mixed bone fill material into the implant area. The rotating mixing component may be oval shaped. The motor may be a reduction motor and may be controlled remotely via a wired or wireless system.

In one embodiment, the injector comprises a force applicator component, with a proximal end and a distal end, comprising a main body, a piston slidably disposed within the main body, and a motor; and wherein the force applicator component is controlled by a remote control device; a delivery reservoir component comprising a reservoir and an outlet, wherein the delivery reservoir component is releasably connected to the force applicator component. Preferably, the piston moves along a central axis within the main body of the injector. Additionally, it is preferable when the movement of the piston is powered and/or controlled by the motor placed at or near the proximal end of the force applicator, wherein the motor is connected to the piston, and wherein a gasket connects the piston to delivery reservoir at the distal end of the force applicator. Additionally, it is preferable when the motor generates a force which is transferred to the piston for ejecting the bone fill material through the delivery outlet into an implant area.

In alternative embodiments of the method for injecting bone fill material, the motor includes an electric motor. In another embodiment, the injector injects the bone fill material where at least one of the volume and flow rate of injected cement is accurately controlled via the motor. In another embodiment, a remote control device controls the motor via at least one of a wire and wireless connection.

In alternative embodiments of the method for injecting bone fill material, the remote control comprises one or more (e.g., 2, 3, 4, 5, 6, or more) of a first screen for showing the time spent and remaining for injecting bone fill material; a second screen for showing the cumulative volume of the injected bone fill material; a button for injecting low volume of bone fill material; a button for injecting moderate volume of bone fill material; a button for injecting high volume of bone fill material; a button of departure countdown; a switch button to turn on or off the remote control; a light to indicate that transfer has begun; a light to indicate that injection has begun; a light to indicate that injection has ended; and a button to stop injection in an emergency. In another embodiment, the motor is connected to the piston with circlips. In another embodiment, the delivery outlet is in fluid communication with a cannula or a needle. In another embodiment, the shape of the force applicator component may be cylindrical.

In some embodiments of the method for injecting bone fill material, the mixer comprises two or more of a mixing reservoir; a rotating mixer; a crank; and a cover; wherein the crank is connected at one end to the handle and at the other end to the rotating mixer, and the rotating mixer is rotated with the crank for mixing a powder component and a liquid component of the bone fill material in the mixing reservoir. In another embodiment, the mixer further comprises a scraper, wherein the scraper moves along the body of the mixer by rotating along matched threads within the reservoir and is separable from the reservoir by rotating in the reverse direction, and holds down the bone fill material in the reservoir once the cover is removed. Preferably, the rotating mixer creates a shearing effect to mix the powder and liquid components to a homogenous bone fill material mix.

In an alternative specific embodiment of the method for injecting bone fill material, the extractor is used for manual transfer of bone fill material from the mixer to the injector; wherein the extractor comprises a transfer piston, which is assembled to the cement mixing reservoir at distal end and to the force applicator of the injector at proximal end. Preferably, a pressure on the upper edge of the transfer piston causes the piston to descend into the mixing reservoir, thereby displacing the cement upwards into the main body of the force applicator.

In some embodiments of the disclosure, a device is provided for delivering flowable materials into a vertebrate body. The device may include an injector, which may have: 1) a delivery reservoir component and 2) a force applicator component. The delivery reservoir component may have a proximal end for receiving the force applicator component and distal end comprising a delivery outlet.

The delivery reservoir component may house flowable materials before and during injection into a vertebrate body. The delivery reservoir component may be oval shaped or cylindrical.

According to some embodiments of the disclosure, the force applicator component houses a force applicator used to generate and/or apply a force to drive the flowable materials, housed in delivery reservoir, through the delivery outlet connected an the distal end of the delivery reservoir. The force applicator component may be cylindrical in shape and may be a hollow barrel, where a force applicator for generating and propagating the force for delivery may be housed. The force applicator may be connected (e.g., via circlips) to the housing of the force applicator component. The force applicator may be a motor or engine. The force applicator may be a reduction motor or engine.

In some embodiments, a force applicator generates a force transferable to the force propagating component (e.g., a piston or plunger) for delivery of flowable materials, housed in the delivery reservoir, through the delivery outlet. The force applicator may be a device that may be operated without any physical contact with the operator.

According to some embodiments, the force applicator housed in the force applicator component may be controlled remotely. In some embodiments, the force applicator may be operated with a remote control device, where the remote control allows an operator to perform the delivery function away from any harmful exposure.

In some embodiments of the disclosure, the remote control may control the volume and speed of delivery of the flowable material, and may allow the operator to precisely control the delivery of the material. The delivery of the bone fill material is controlled with the remotely controlled force applicator, which transfers the force generated to the propagating means (for example: a piston), thus ensuring that the volume and flow rate of injected cement is accurately controlled and precisely delivered in a vertebrate body, and without exposing the operator of the device to harmful byproducts generated during the procedure.

According to some embodiments of the disclosure, the delivery outlet is connected to a delivery channel, where one end of the delivery channel is connected to the delivery outlet of the injector. The other end of the delivery channel may be placed at or into the delivery site (e.g., within an vertebral body). The delivery channel may be tubular. The delivery channel may be rigid or flexible. In some embodiments, the delivery channel is a cannula or syringe needle.

The delivery reservoir, in some embodiments of the disclosure, may be coupled to a transfer apparatus (e.g., extractor), prior to its coupling with the injector. The transfer apparatus allows manual transfer of flowable material into the injector. In some embodiments, the transfer apparatus allows manual transfer of flowable material into the injector from a mixing apparatus, where the flowable materials may be mixed before or during transferring.

According to some embodiments of the disclosure, the mixing apparatus may have or be coupled to the mixing reservoir or well, where components of the flowable materials may be mixed. The mixing apparatus may also have a rotating mixer, a cover, and a scraper. The rotating component may be have a crank and a handle, in that the crank is rotated with the handle to mix components of the flowable material in the mixing reservoir or well. The mixing reservoir or well may have a cylindrical shaped body, where inside the hollow chamber the components of the flowable material may be mixed by rotating the crank.

In some embodiments, the flowable material may be bone fill material, which when delivered into a vertebrate body, may serve as a junction between the bone and the implant, and is non-toxic and biocompatible. In further embodiments, the bone fill material is prepared by mixing a powder component and a liquid component in the mixing reservoir or well of the mixing device, subsequently manually transferred to the delivery reservoir component injector with the extractor, which is then connected to the force applicator component to form the injector, and delivered through the delivery outlet of the injector.

According to some embodiments, the bone fill material has a viscosity of between about 1 to 2000 Pa·s. In some other embodiments, the bone fill material has a viscosity, between 500-2000 Pa·s.

### BRIEF DESCRIPTION OF THE DRAWINGS

FIGS. 1A-1D are illustrations of a Mixer device (which may be part of a mixing and injection system) according to some embodiments.

FIGS. 2A-2D are illustrations of an Extractor device/component (which may be part of a mixing and injection system) according to some embodiments.

FIG. 3A-3D are illustrations of an Injector device/component for a mixer (which may be part of a mixing and injection system), according to some embodiments.

FIG. 4 displays a control device useful for remotely controlling bone fill material injection.

### DETAILED DESCRIPTION OF EMBODIMENTS OF THE DISCLOSURE

In the following discussion, the terms “proximal” and “distal” will be used to describe the opposing axial ends of the inventive injector device, as well as the axial ends of various component features. The term “proximal” is used in its conventional sense to refer to the end of the apparatus (or component thereof) that is closest to the operator during use of the apparatus. The term “distal” is used in its conventional sense to refer to the end of the apparatus (or component thereof) that is initially inserted into the patient, or that is closest to the patient.

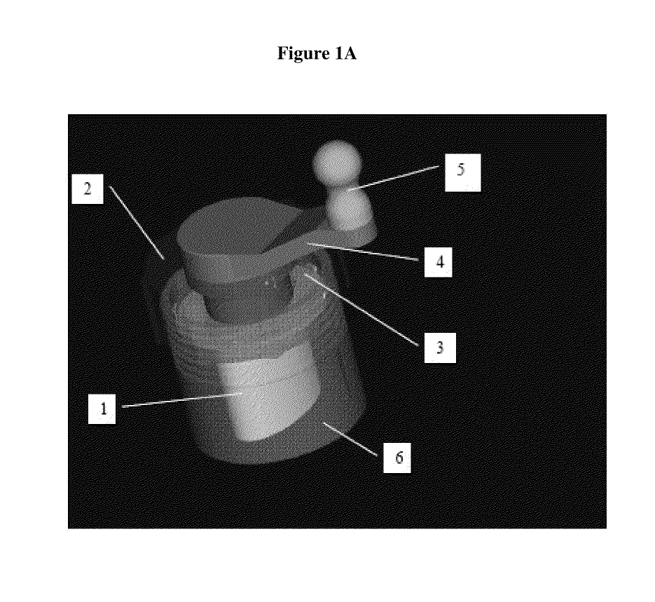

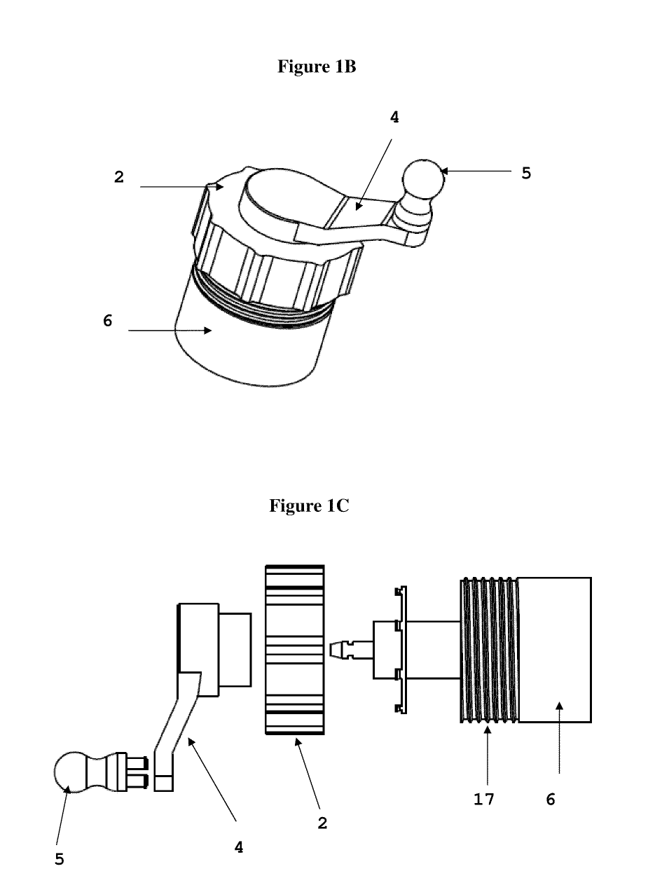

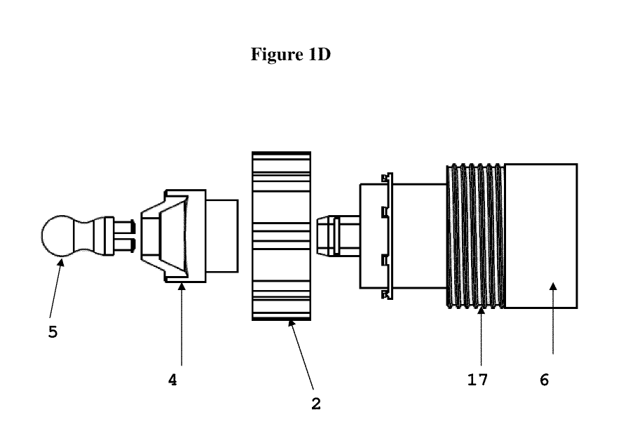

Certain embodiments of the invention provide systems and methods for treating bone, such as a vertebra, by delivering bone fill material into the interior of the vertebra. FIGS. 1-3 illustrate a mixing and delivery system for mixing and/or delivery of a bone fill material, in accordance with some embodiments of the disclosure.

According to some embodiments of the disclosure, an injector device/component may be provided for injecting bone fill material into a vertebrate body. According to further embodiments, the device/component(s) of the present invention include a three piece unit with interlocking components, where parts of each may be replaced with extraneous parts to alternatively form any one of the three components described in the current invention.

### Injector

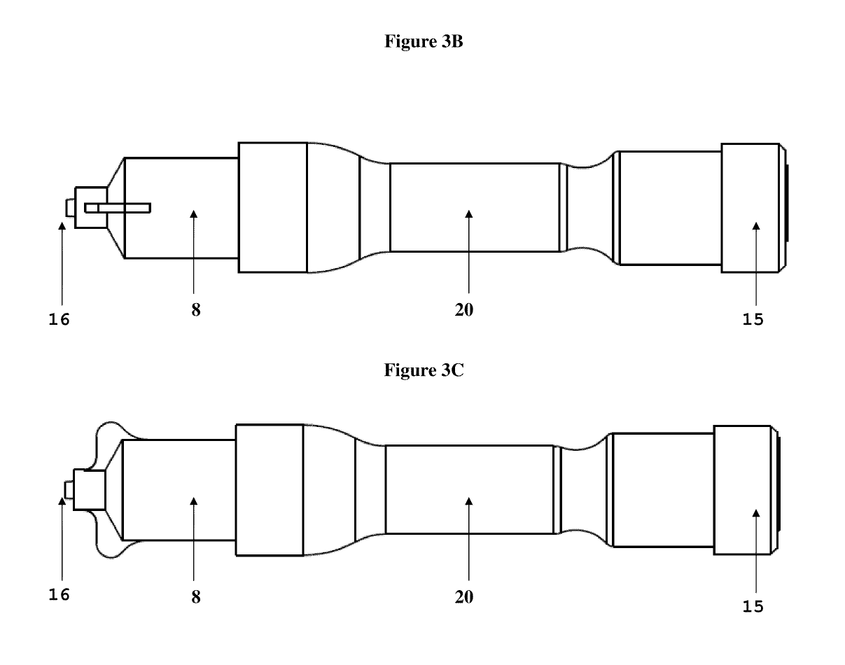

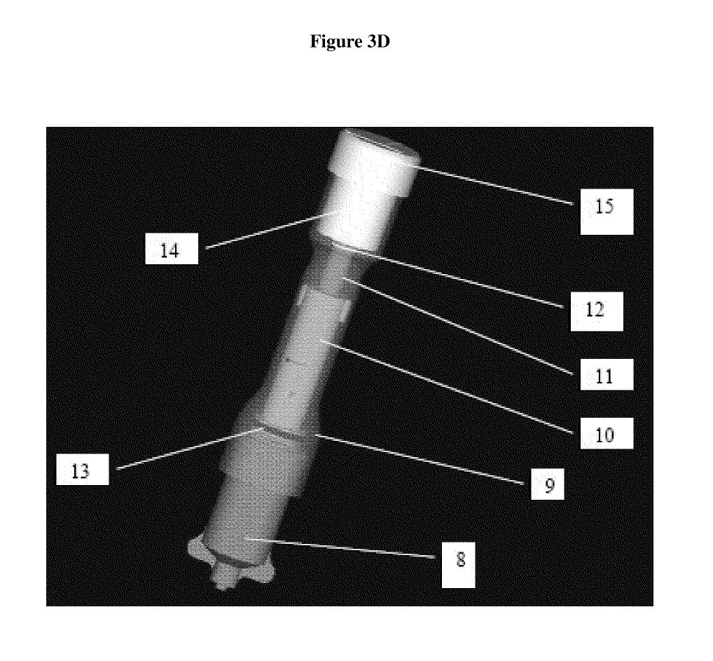

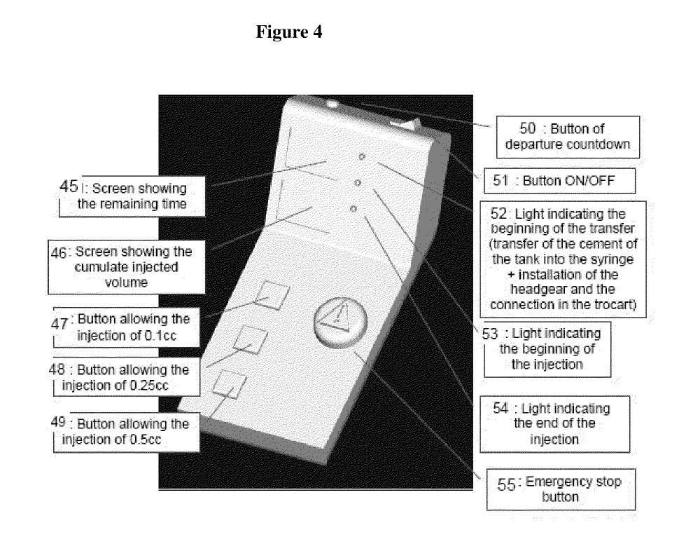

FIGS. 3A-3D provide illustrations of an injector according to some embodiments. In some embodiments, the injector comprises 1) a delivery reservoir component 8 and 2) a force applicator component 9. The delivery reservoir component is releasably connected to the force applicator component 20. The delivery reservoir component 8 has a proximal end for receiving the force applicator 20 component and a distal end having a delivery outlet 16. The delivery reservoir component 8 houses the flowable materials or bone fill material before and during injection into a vertebrate body.

In some embodiments of the disclosure, a delivery reservoir component 8 is connected to the force applicator component 20 at the distal end of the injector force applicator component 20. For example, the main body 9 of the force applicator component 20 is designed to receive the proximal end of the a delivery reservoir component 8. Any connection mechanism may be used to connect the force applicator component 20 with the delivery reservoir component 8. For example, the distal end of the delivery reservoir component 8 and the distal end of the main body 9 of the force applicator component 20 may be threaded such that the force applicator component 20 is screwed onto the delivery reservoir component 8 thereby forming the injector device. See FIG. 3D.

In some embodiments of the disclosure, the delivery reservoir component 8 is cylindrical shaped. In yet in other embodiments, the delivery reservoir component 8 is oval shaped, or polygon shaped, or other geometric shapes that provides sufficient space for mixing bone fill material. In some embodiments, the delivery reservoir component 8 may be oval shaped. In some embodiments, the delivery reservoir component 8 may be in the form and share of a syringe.

In some embodiments of the disclosure, the force applicator component 20, with a proximal end and a distal end, may include a main body 9, a piston or plunger slidably disposed within the main body 10, and a force applicator (e.g., motor) 14. The force applicator component 20 may further comprise a cover 15 at the proximal end to shield the force applicator 14. The force applicator 14 may be an electric motor, a reduction motor, a hand crank, or other mechanism capable of transmitting hand or powered energy to the piston 10. The piston 10 is connected to the force applicator 14 via a connection means 12. In some embodiments, the force applicator 14 may be detached but connected with wires to the piston 10. The force applicator component 20, may be controlled by a remote control device. The force applicator 14 (e.g., electric motors) may be housed at the proximal end of the force applicator component 20.

In some embodiments of the disclosure, the injector utilizes a force applicator 14 (e.g., an electric motor, a hand-crank, and the like) to power a piston for generating the force for injecting bone fill material into a vertebrate body. The piston 10 may be placed within the main body 9 or barrel of the injector and may be powered by a force applicator 14, enabling regulated injection of bone fill material through an outlet (e.g., cannula or a syringe needle).

The open end of the delivery outlet 16 may be fitted with a hypodermic needle, or a nozzle, or a tubing to help direct the flow into and out of the barrel. The barrel of the syringe may be made of plastic or metal or glass. The delivery outlet 16 may be connected to a cannula or syringe needle. When the force applicator is on, the piston moves along a central axis 11 (i.e., axis of training) and applies a force on the bone fill material contained in the delivery reservoir 8. The bone fill material is then expelled through the delivery outlet 16, into the cannula or syringe needle, and out through the orifice at the open end of the cannula or syringe needle.

The size and shape of the reservoir 8 may be altered to accommodate a reduced or increased volume. According to some embodiments, the reservoir 8 may be loadable with sufficient bone fill material to treat one vertebra with a single injected aliquot. In such embodiments, the reservoir 8 will have a size and shape appropriate for a single injected aliquot. In other embodiments, the reservoir 8 may be loadable with sufficient bone fill material to treat two or more (3, 4, 5, 6, 7, 8, 9, etc.) vertebra.

According to some embodiments, the reservoir 8 may be loadable with from about 0.5 ml to 20 ml of bone fill material, preferably from about 5 to about 10 ml (e.g., about 5.5 ml, 6 ml, 7 ml, 8 ml, 9 ml) of bone fill material. In some embodiments, the reservoir 8 is usable with volumes of less than 5 ml (e.g., 1, 2, 3, 4), or greater than 10 ml in volumes. In some embodiments, delivery reservoir 8 may be loadable with sufficient bone fill material to treat at least two vertebrae, or at least 3, or at least 4 vertebrae without refilling. This may reduce the number of access procedures for each vertebra, to a single access procedure.

In some embodiments, the low volume capacity of bone fill material for the reservoir 8 is approximately between 0.01-0.24 cc, approximately between 0.02-0.24 cc, 0.03-0.24, 0.04-0.24 cc, 0.05-0.24 cc, 0.06-0.24 cc, 0.07-0.24 cc, 0.08-0.24 cc, 0.09-0.24 cc, 0.1-0.24 cc. According to some embodiments, the volume capacity of bone fill material for the reservoir 8 is 0.1 cc. In some embodiments, the volume capacity of bone fill material for the reservoir 8 is approximately between 0.25-0.49 cc, approximately between 0.25-0.48 cc, 0.25-0.47, 0.25-0.46 cc, 0.25-0.45 cc, 0.25-0.44 cc, 0.25-0.43 cc, 0.25-0.42 cc, 0.25-0.41 cc, 0.25-0.40 cc, 0.25-0.39 cc, 0.25-0.38 cc, 0.25-0.37 cc, 0.25-0.36 cc, 0.25-0.35 cc, 0.25-0.34 cc, 0.25-0.33 cc, 0.25-0.32 cc, 0.25-0.31 cc, 0.25-0.30 cc. According to some embodiments, the volume is 0.25 cc. In some embodiments, the volume capacity of bone fill material for the reservoir 8 may be approximately between 0.5-1.00 cc, approximately between 0.5-0.9 cc, 0.5-0.8, 0.5-0.7 cc, or 0.5-0.6 cc. According to some embodiments, the volume capacity is 0.5 cc.

The injector may be constructed of amorphous nylon, for e.g. Durethan® (LANXESS, Leverkuzen, Germany), Grilamid® (EMS-GRivory, Reichenauerstras se, Switzerland) or Topas® (Ticona GmbH, Kelsterbach, Germany). In some embodiments of the invention, the injector may be constructed of polycarbonate or other material as known in the art the time of invention. The material selected may be resistant to corrosion from the cement, and may be transparent. In some embodiments, the thickness of walls of the main body of the injector may be greater than 3 mm, or 4 mm, or 5 mm, or 6 mm, or any values intermediary or less than 3 mm. According to some embodiments, the main body of the injector is characterized by a wall thickness to internal diameter ratio of about 0.23, or 0.25, or 0.27, or 0.29 (e.g., wall thickness of about 5 mm and internal diameter of about 18 mm). The ratio may provide sufficient strength to withstand pressures of about 100 to 300 atmosphere.

### Force Applicator

According to some embodiments, force applicator 14 may be coupled to a piston, 10, which provides the requisite torque and speed to push the piston, allowing precise regulation of pressure and/or flow rate used to deliver the bone fill material into a vertebrate body.

In some embodiments, the force applicator 14 drives a threaded part (e.g., a threaded rod) 11 with a specific shape. This part drives the piston 10 in translation, since the piston is blocked in rotation due to two tongues or grooves within the main body of the injector. The speed of the force applicator may be fixed at a very low rotation speed. The rotation speed may be between approximately 0.5-15 rpm, or may be between approximately 6-14 rpm, between approximately 7-13 rpm, between approximately 8-12 rpm, between approximately 9-11 rpm. In some embodiments, the speed of rotation is approximately 7 rpm.

According to some embodiments, the force applicator 14 may be a reduction motor that can be operated remotely to precisely control the volume and flow-rate of injection.

In some embodiments of the disclosure, the force applicator is a motor. The motor may be electric. In some embodiments, a power source (AC or DC) supplies power to the force applicator. In some embodiments, a battery(s) may be used to supply power.

An AC motor may be a synchronous motor or an induction motor or an commutator motor. The synchronous motor may be a plain synchronous motor or a super synchronous motor. The induction motor may be a squirrel cage rotor or a slip ring motor. The commutator motor may be a series motor or a shunt motor or a compensating motor, or a repulsion motor, or a repulsion induction motor.

A DC motor may be brushed DC electric motor or a brushless DC motor. The brushless DC motor may a stepper motor or a reluctance motor. The stepper motor may be a permanent magnet stepper, or a hybrid synchronous stepper, or a variable reluctance stepper.

According to some embodiments of the disclosure the motor may be an AC parallel shaft gear-motor, or a AC right angle gear-motor, or a DC parallel shaft gear-motor, or DC right angle gear-motor, or a hollow shaft gear-motor. The gear-motor of the disclosure may have a speed reducer, which may be an angle speed reducer.

In further embodiment, the force applicator (e.g., an electric motor, a hand-crank, and the like) may be operated from outside the room via a remote control (wired and/or wireless), where the procedure is being done, e.g., from an observation area, thereby reducing exposure of medical personnel to adverse radiation, such as X-ray, and/or other procedural contaminants. Once sufficient or desired bone fill material is delivered into the vertebra or other body parts, the needle or cannula attached to the injector may be removed and the puncture or other access opening may be closed using state of the art procedures at the time of invention or performing the procedure.

### Piston

According to some embodiments of the disclosure, the piston 10 may be inserted into the proximal end of the injector or otherwise slidably disposed within the main body 9, and powered by force applicator 14. The piston may form a circumferential seal with respect to the inner surface of the main body of the injector. In some embodiments of the disclosure, the piston 10 may be advanced distally within the main body of the injector 9 and into the delivery reservoir component 8, thereby applying a force creating sufficient pressure to inject the compound (e.g., bone fill material) stored within the delivery reservoir 8 and out through the delivery outlet 16.

In some embodiments of the disclosure, piston may include a nipple (not shown) extending into the main body cavity 9. The nipple may have a size corresponding to the outlet port of the delivery outlet 8, e.g., such that the nipple may be slidably received in the outlet port of the delivery outlet 8 as the piston 10 is depressed distally. This may minimize the amount of bone fill material remaining within the delivery outlet 8 when the piston is fully depressed.

In some embodiments of the disclosure, the piston may be a power piston or a displacement piston.

A plunger or other device may be placed at the distal end of the piston. The plunger comes into contact with the bone fill material as the expulsion force is applied. In some embodiments, the plunger or other expulsion means forms a circumferential seal with respect to the inner surface of the delivery reservoir 8, such as in a syringe, as the plunger is forced toward the distal end of the injector.

### Snap Rings and Gasket

In some embodiments, snap rings 12 connect the force applicator to the piston on a proximal end and a gasket 13 is placed connects the piston to syringe at the distal end. According to some embodiments, the snap rings are circlips. In such embodiments, circlips 12 may connect the force applicator to the piston on a proximal end and the gasket 13 connects the piston to syringe at the distal end. According to some embodiments, the circlips are external metric circlip 12. The external circlip is fixed onto the piston 10 in the main body. According to some embodiments, circlips are internal metric circlip.

### Remote Control

In some embodiments, a remote controls may be used to issue commands from a distance to the force applicator (electric motor). Remote controls for these devices may be wireless handheld objects with an array of buttons and screens for adjusting various settings such a volume and flow rate. The remote device communicate to their electric motor via infrared (IR) signals and a few via radio signals. The remote control device may be powered by battery (for e.g., AAA or AA size batteries).

In some embodiments, the remote control comprises: a) a first screen 45 for showing the time spent and remaining for injecting bone fill material; b) a second screen 46 for showing the cumulative volume of the injected bone fill material; c) a button 47 for injecting low volume of bone fill material; d) a button 48 for injecting moderate volume of bone fill material; e) a button 49 for injecting high volume of bone fill material; f) a button 50 of departure countdown; g) a switch 51 button to turn on or off the remote control h) a light 52 indicating that transfer has begun; i) a light 53 indicating injection has begun; j) a light 54 indicating injection has ended; and k) a button 55 to stop injection in an emergency.

According to some embodiments, the remote control may be used in the following chronology: a) the switch button 51 (on/off) is a tongue which is turned to the “on” position; b) the mixture is manually initiated to mix powder and liquid components of bone fill material, and simultaneously the operator of the injector device presses on the button of departure countdown 50 to start the process of injection; c) at approximately 2 minutes later, the light indicating the beginning of the transfer 52 of bone fill material is lit, which indicates that the transfer of bone fill material from the mixer to the injector need to begin; d) another 2 minutes later, when the transfer is over, the second light comes on 53 to indicate that the injection should be started; e) operator may choose the volume of the injected bone fill material using the buttons for low, moderate or high volume of injection, using the buttons 47-49; f) if a leak occurs, the operator can press the emergency stop button 55; g) at the end of the countdown, the last of the three lights 54 comes on to indicate that the injection must be stopped at the earliest; and h) once the injection is over the switch button 51 (on/off) is turned to the off mode.

In further embodiments, the chronology of injection with the remote control may take approximately less than 30 minutes, or between 5-20 minutes, or between 5-15 minutes, or between 10-15 minutes.

According to some embodiments, chronology of injection with the remote control may take approximately 12 minutes, and a screen on the remote control may indicate the beginning time at 12 minutes, and count down during the process. The operator (for example, a surgeon) may be able to check the remaining time during the surgery. A second screen may also be present, where the volume of the injected bone fill material is displayed. The number of turns of the motor controls the volume injected during surgery.

### Mixer

According to some embodiments, the mixer component may include any one or more of: (i) a mixing reservoir 6 adapted to contain at least a polymer component and a monomer component of the bone fill material to be mixed; (ii) a rotating mixer 1 attachable to a crank 4 (iii) a crank 4 adapted to move the rotating mixer along the travel path in the mixing tank so that a reference point on the mixer will face in the same direction at every point on travel path; (iv) a handle 5 for moving the crank; (v) a cover 2 and (iv) a scraper. The mixing reservoir 6 may be a cylindrical shaped body. The powder and the liquid components of the bone fill material may be mixed using a rotating mixer 1. The rotating mixer may be oval shaped; the oval shape is useful for mixing powder and liquid components of bone fill material because it creates a shearing effect. The crank 4 may be connected at one end to the handle 5 and at the other end to the rotating mixer 1. The bone fill material, after mixing, is homogenous and viscous, which may be held down in the reservoir with a scraper, when the cover of the reservoir is removed.

According to some embodiments of the disclosure, the mixer may have a large surface area of about 400 mm², or more than 400 mm², or more than 500 mm², or more than 600 mm², or more than 800 mm², or more than 1000 mm².

In some embodiments of the disclosure, cover may be closed by lowering it onto the reservoir, so that tabs (not shown) are engaged by slots (not shown), thus preventing the rotation of cover with respect to the reservoir. In some embodiments, other rotational locking components may be employed that are in the state of the art at the time of the invention.

Revolution of handle may turn axle (not shown) and may cause revolution of gears (not shown). In some embodiments of the disclosure, the axle (not shown) may be rotated by an electric motor. The electric motor may be battery powered or AC powered. In some embodiments of the disclosure, the axle may be rotated with hand.

Rotating mixer may be attached via an axle to gear located on revolving plate. Axle, when turned, may cause revolution of the gears (not shown). Revolution of revolving plate may cause axle of the rotating mixer to revolve about a center of the reservoir, inducing a shearing effect for preparing the homogenous mix of the bone fill material.

The mixer may be constructed of a wide variety of materials. In some embodiments of the disclosure, the choice of construction materials may consider the type of bone fill material or viscous biomaterial to be mixed, its chemical characteristics and/or viscosity. Mixing reservoir may be constructed at least partially of polypropylene and/or nylon. According to some embodiments, the rotating mixer may be constructed of stainless steel, or plastic and/or other metal.

According to some embodiments of the disclosure, when the mixing of the powder and liquid components of bone fill material to a homogenous viscous composition is accomplished, the cover may be opened, and the scraper may then hold the viscous bone fill material in the reservoir until the transfer is initiated with the extractor.

### Extractor

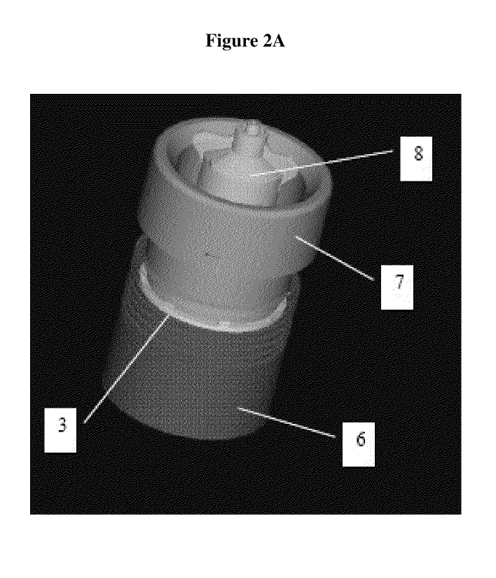

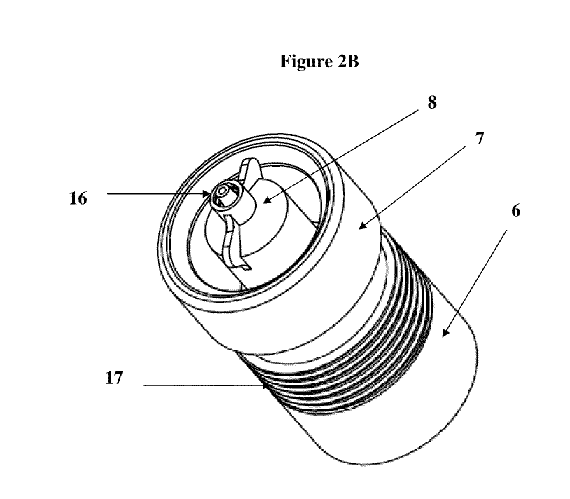

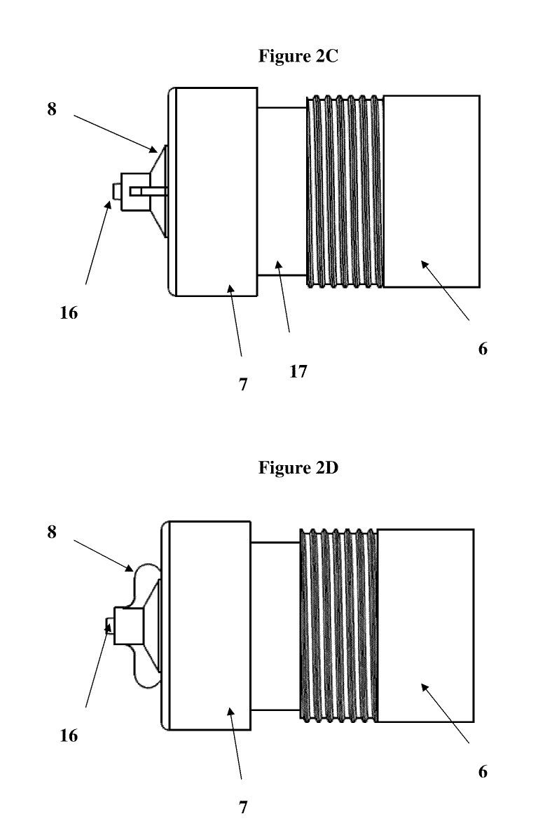

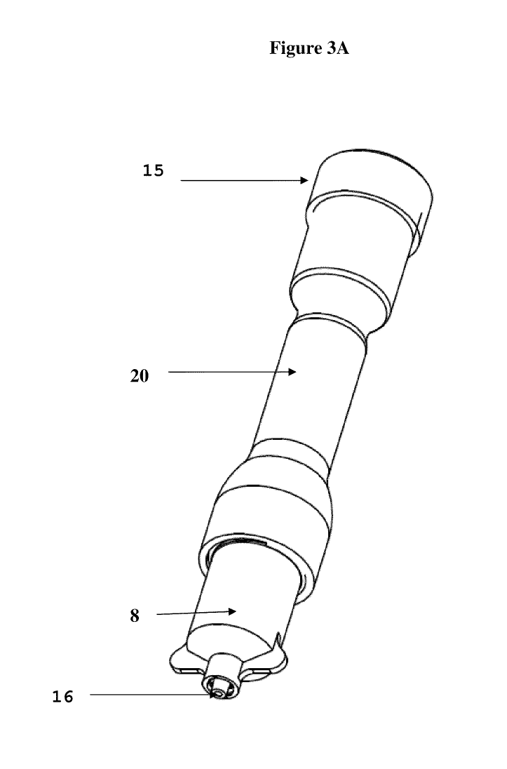

FIGS. 2A-2D illustrate an embodiment of an extractor for loading a viscous material into a container. According to some embodiment of the disclosure, the material is a viscous bone fill material. In some embodiments of the disclosure, the extractor may have one or more of the following: (i) scraper 3; (ii) reservoir 6; and (iii) a delivery reservoir component 8 (e.g., syringe) (shown in FIG. 2).

According to some embodiments of the disclosure, the extractor component is used to move the bone fill material from the reservoir/tank 6 of the mixer to the injector. The purpose of the extractor is to manually transfer bone fill material from the mixing reservoir for delivery using the injector. In some embodiments, the mixing reservoir and the delivery reservoir may be the same, however, designated with different names so depending on which component (mixer or the injector) of the bone injection system it is attached to. Thus, the transfer of the bone fill material may be manually achieved by moving the bone fill material from the reservoir of the mixer to the delivery reservoir component 8, assisted by the extractor. The bone fill material may be retained in the reservoir for delivery, and when pressure is generated, using the force applicator and through the piston within the injector, the bone fill material is delivered through the delivery outlet component (such as a syringe) of the injector.

According to some embodiments of the disclosure, the extractor may be attached to a transfer piston, which may be used for transfer of the bone fill material from the reservoir into the main body of the injector. Application of pressure to reservoir and/or an upper edge of transfer piston causes piston to descend into container/reservoir. Cement is thereby displaced upwards from the reservoir to the main body of the injector. When the main body of the injector is sufficiently filled, it is separated from the transfer piston of the extractor.

Assembly of the reservoir and transfer piston may be via mated threads of reservoir and transfer piston. The cover may be employed to force transfer piston downwards into the mixing reservoir, the cover may be threaded on the reservoir via complementary threads. In some embodiments of the disclosure, the cover may be screwed onto the reservoir to force piston downwards onto the cement. The downward motion of the piston may force the cement to rise upwards into the main body of the injector.

### Kits

Some embodiments of the present disclosure provides for kits and devices for injecting bone fill material in a vertebral body, which may comprise one or more of, and in some embodiments, all of: (i) an injector; (ii) a mixer; and (iii) an extractor.

Some embodiments of the present disclosure provides for kits and devices for injecting bone fill material in a vertebral body, which may comprise one or more of, and in some embodiments, all of: (i) a mixer device/component, wherein the mixer component is used to mix a powder component and a liquid component of a bone fill material; (ii) an extractor device/component, wherein the extractor component is used for transferring the mixed bone fill material from the mixing tank into the drug reservoir component; (iii) an injector device/component, wherein the injector component is used for injecting the mixed bone fill material from the drug reservoir component into a part of the vertebral body, for e.g., the vertebra.

### Bone Fill Material

“Bone fill material, infill material or composition” includes its ordinary meaning and is defined as any material for infilling a bone that includes an in-situ hardenable material, such as bone cement. The fill material also can include other “fillers” such as filaments, microspheres, powders, granular elements, flakes, chips, tubules and the like, autograft or allograft materials, as well as other chemicals, pharmacological agents or other bioactive agents.

“Flowable material” includes its ordinary meaning and is defined as a material continuum that is unable to withstand a static shear stress and responds with an irrecoverable flow (a fluid)—unlike an elastic material or elastomer that responds to shear stress with a recoverable deformation. Flowable material includes fill material or composites that include a fluid (first) component and an elastic or inelastic material (second) component that responds to stress with a flow, no matter the proportions of the first and second component, and wherein the above shear test does not apply to the second component alone.

“Vertebroplasty” includes its ordinary meaning and means any procedure wherein fill material is delivered into the interior of a vertebra.

According to some embodiments of the disclosure, methods are provided for flowing a viscous cement from a reservoir or tank 6 into a bone via an injector, the method comprising: generating a pressure within a cement having a viscosity of at least 900 Pascal seconds and residing in a reservoir or tank 6 in response to an actuation input; wherein the force is generated using force applicator 14 powered piston 10 and forces at least some of the cement out of the reservoir through the outlet syringe 8.

According to some embodiments, the viscosity of the bone fill material may be at least 500 Pascal-second. In some embodiments, bone fill material achieves a viscosity of at least 500 Pascal-second within 120 seconds following initiation of mixing of monomer and polymer components.

In some embodiments, bone fill material achieves a viscosity of at least 500 Pascal-second within 60 seconds following initiation of mixing of monomer and polymer components. In some embodiments, bone fill material achieves a viscosity of at least 500 Pascal-second within 45 seconds following initiation of mixing of monomer and polymer components. In some embodiments, bone fill material achieves a viscosity of at least 500 Pascal-second within 30 seconds following initiation of mixing of monomer and polymer components. In some embodiments, bone fill material achieves a viscosity of at least 500 Pascal-second within 15 seconds following initiation of mixing of monomer and polymer components.

In some embodiments, viscosity of the bone fill material changes less than 10% within subsequent 2 minutes. In some embodiments, viscosity of the bone fill material changes less than 20% within subsequent 2 minutes.

According to some embodiments of the disclosure, the viscosity of the bone fill material is about 400 Pascal-sec.; or about 400-450 Pascal-sec.; or about 450-500 Pascal sec.; or about 500-550; or about 550-600 Pascal sec.; or about 600-650 Pascal sec.; or about 650-700 Pascal sec.; or about 700-750 Pascal sec.; or about 750-800 Pascal sec.; or about 800-850 Pascal sec.; or about 850-900 Pascal sec.; or about 900-950 Pascal sec.; or about 950-1000 Pascal sec.; or about 1000-1100 Pascal sec.; or about 1100-1200 Pascal sec.; or about 1200-1300 Pascal sec.; or about 1300-1400 Pascal sec.; or about 1400-1500 Pascal sec.; or about 1500-1600 Pascal sec.; or about 1600-1700 Pascal sec.; or about 1700-1800 Pascal sec.; or about 1800-1900 Pascal sec.; or about 1900-2000 Pascal sec. In a preferred embodiment the viscosity is within 500-2000 Pascal sec. range.

### Specific Embodiments

A bone fill material injection system for injecting bone fill material, by remote control, into an area of bone, the system comprising: an injector, where the injector comprises: (i) a force applicator component, with a proximal end and a distal end, having the following components: a main body, a piston slidably disposed within the main body, and a motor; and wherein the force applicator component is controlled by a remote control device; (ii) a delivery reservoir component comprising a reservoir and an outlet, where the delivery reservoir component is releasable connected to the force applicator component.

The bone fill material injection system, in which the piston moves along a central axis within the main body of the injector.

The bone fill material injection system, in which movement of the piston is powered and/or controlled by the motor placed at or near the proximal end of the force applicator, wherein the motor is connected to the piston, and wherein a gasket connects the piston to delivery reservoir at the distal end of the force applicator.

The bone fill material injection system, in which the motor generates a force which is transferred to the piston for ejecting the bone fill material through the delivery outlet into an implant area.

The bone fill material injection system, in which the motor comprises a motor with a reducer.

The bone fill material injection system, in which the motor includes an electric motor.

The bone fill material injection system, in which the injector injects the bone fill material, and wherein at least one of the volume and flow rate of injected cement is accurately controlled via the motor.

The bone fill material injection system, in which the remote control device controls the motor via at least one of a wire and wireless connection.

The bone fill material injection system, in which the remote control comprises one or more of the following: a) a first screen for showing the time spent and remaining for injecting bone fill material; b) a second screen for showing the cumulative volume of the injected bone fill material; c) a button for injecting low volume of bone fill material; d) a button for injecting moderate volume of bone fill material; e) a button for injecting high volume of bone fill material; f) a button of departure countdown; g) a switch button to turn on or off the remote control; h) a light to indicate that transfer has begun; i) a light to indicate that injection has begun; j) a light to indicate that injection has ended; and k) a button to stop injection in an emergency.

The bone fill material injection system, in which the motor is connected to the piston with circlips.

The bone fill material injection system, in which the delivery outlet is connected to a cannula or a syringe needle.

The bone fill material injection system, in which the shape of the force applicator component is cylindrical.

The bone fill material injection system, in which a mixer is include, comprising two or more of the following: (i) a mixing reservoir; (ii) a rotating mixer; (iii) a crank; and (iv) a cover; wherein the crank is connected at one end to the handle and at the other end to the rotating mixer, and the rotating mixer is rotated with the crank for mixing a powder component and a liquid component of the bone fill material in the mixing reservoir.

The bone fill material injection system, in which the mixer further comprises a scraper, wherein the scraper moves along the body of the mixer by rotating along matched threads within the reservoir and is separable from the reservoir by rotating in the reverse direction, and holds down the bone fill material in the reservoir once the cover is removed.

The bone fill material injection system, in which the rotating mixer is oval shaped, which creates a shearing effect to mix the powder and liquid components to a homogenous bone fill material mix.

The bone fill material injection system, in which an extractor is used for manual transfer of bone fill material from the mixer to the injector. The extractor comprises a transfer piston, which is assembled to the cement mixing reservoir at distal end and to the force applicator of the injector at proximal end.

The bone fill material injection system, in which a pressure on the upper edge of the transfer piston causes the piston to descend into the mixing reservoir, thereby displacing the cement upwards into the main body of the force applicator.

A method for injecting bone fill material into an implant area comprising: mixing a powder component and a liquid component of bone fill material together in a mixer, using an extractor to transfer the mixed bone fill material from the mixer to an remotely controlled engine powered injector; and injecting the mixed bone fill material into the implant area.

The method for injecting bone fill material, in which the injector comprises: (i) a force applicator component, with a proximal end and a distal end, comprising: a main body, a piston slidably disposed within the main body, and a motor; and wherein the force applicator component is controlled by a remote control device; (ii) a delivery reservoir component comprising a reservoir and an outlet, wherein the delivery reservoir component is releasable connected to the force applicator component.

The method for injecting bone fill material, in which the piston moves along a central axis within the main body of the injector.

The method for injecting bone fill material, in which movement of the piston is powered and/or controlled by the motor placed at or near the proximal end of the force applicator, wherein the motor is connected to the piston, and wherein a gasket connects the piston to delivery reservoir at the distal end of the force applicator.

The method for injecting bone fill material, in which the motor generates a force which is transferred to the piston for ejecting the bone fill material through the delivery outlet into an implant area.

The method for injecting bone fill material, in which the motor comprises a motor with a reducer.

The method for injecting bone fill material, in which the motor includes an electric motor.

The method for injecting bone fill material, in which the injector injects the bone fill material, and wherein at least one of the volume and flow rate of injected cement is accurately controlled via the motor.

The method for injecting bone fill material, in which the remote control device controls the motor via at least one of a wire and wireless connection.

The method for injecting bone fill material, in which the remote control comprises one or more of the following: a) a first screen for showing the time spent and remaining for injecting bone fill material; b) a second screen for showing the cumulative volume of the injected bone fill material; c) a button for injecting low volume of bone fill material; d) a button for injecting moderate volume of bone fill material; e) a button for injecting high volume of bone fill material; f) a button of departure countdown; g) a switch button to turn on or off the remote control; h) a light to indicate that transfer has begun; i) a light to indicate that injection has begun; j) a light to indicate that injection has ended; and k) a button to stop injection in an emergency.

The method for injecting bone fill material, in which the motor is connected to the piston with circlips.

The method for injecting bone fill material, in which the delivery outlet is in fluid communication with a cannula or a needle.

The method for injecting bone fill material, in which the shape of the force applicator component is cylindrical.

The method for injecting bone fill material, in which the mixer comprises two or more of the following: (i) a mixing reservoir; (ii) a rotating mixer; (iii) a crank; and (iv) a cover; wherein the crank is connected at one end to the handle and at the other end to the rotating mixer, and the rotating mixer is rotated with the crank for mixing a powder component and a liquid component of the bone fill material in the mixing reservoir.

The method for injecting bone fill material, in which the mixer further comprises a scraper, wherein the scraper moves along the body of the mixer by rotating along matched threads within the reservoir and is separable from the reservoir by rotating in the reverse direction, and holds down the bone fill material in the reservoir once the cover is removed.

The method for injecting bone fill material, in which the rotating mixer is oval shaped, which creates a shearing effect to mix the powder and liquid components to a homogenous bone fill material mix.

The method for injecting bone fill material, in which the extractor is used for manual transfer of bone fill material from the mixer to the injector; wherein the extractor comprises a transfer piston, which is assembled to the cement mixing reservoir at distal end and to the force applicator of the injector at proximal end.

The method for injecting bone fill material, in which a pressure on the upper edge of the transfer piston causes the piston to descend into the mixing reservoir, thereby displacing the cement upwards into the main body of the force applicator.

While the invention is susceptible to various modifications, and alternative forms, specific examples thereof have been shown in the drawings and are herein described in detail. It may be understood, however, that the invention is not to be limited to the particular forms or methods discloses, but to the contrary, the invention is to cover all modifications, equivalents and alternatives falling within the spirit and scope of the appended claims.

What is claimed is:

1. A bone fill material injection system for injecting bone fill material, by remote control, into an area of bone, the system comprising: an injector, wherein the injector comprises:
(i) a force applicator component, with a proximal end and a distal end, comprising:
a main body, a piston slidably disposed within the main body, and a motor; wherein the force applicator component is controlled by a remote control device; and wherein the motor is a reduction motor; and
(ii) a delivery reservoir component comprising a reservoir and an outlet, wherein the delivery reservoir component is releasably connected to the force applicator component.

2. The system according to claim 1, wherein the remote control device controls the motor via a wire or wireless connection.

3. The system according to claim 1, wherein the remote control comprises one or more of the following:
a) a first screen for showing the time spent and remaining for injecting bone fill material;
b) a second screen for showing the cumulative volume of the injected bone fill material;
c) a button for injecting low volume of bone fill material;
d) a button for injecting moderate volume of bone fill material;
e) a button for injecting high volume of bone fill material;
f) a button of departure countdown;
g) a switch button to turn on or off the remote control;
h) a light to indicate that transfer has begun;
i) a light to indicate that injection has begun;
j) a light to indicate that injection has ended; and
k) a button to stop injection in an emergency.

4. The system according to claim 1, wherein the piston moves along a central axis within the main body of the injector.

5. The system according to claim 4, wherein movement of the piston is powered and/or controlled by the motor placed at or near the proximal end of the force applicator, and wherein the motor is connected to the piston.

6. The system according to claim 5, wherein the motor generates a force which is transferred to the piston for ejecting the bone fill material through the delivery outlet into an implant area.

7. The system according to claim 1, wherein the motor includes an electric motor.

8. The system according to claim 1, wherein the injector injects the bone fill material, and wherein at least one of the volume and flow rate of injected cement is accurately controlled via the motor.

9. The system according to claim 5, wherein the motor is connected to the piston with circlips.

10. The system according to claim 1, wherein the delivery outlet is connected to a cannula or a syringe needle.

11. The system according to claim 1, wherein the shape of the force applicator component is cylindrical.

12. The system according to claim 1, further comprises a mixer, wherein the mixer comprises two or more of the following:
(i) a mixing reservoir;
(ii) a rotating mixer;
(iii) a crank; and
(iv) a cover;
wherein the crank is connected at one end to the handle and at the other end to the rotating mixer, and the rotating mixer is rotated with the crank for mixing a powder component and a liquid component of the bone fill material in the mixing reservoir.

13. The system according to claim 12, wherein the mixer further comprises a scraper, wherein the scraper moves along the body of the mixer by rotating along matched threads within the reservoir and is separable from the reservoir by rotating in the reverse direction, and holds down the bone fill material in the reservoir once the cover is removed.

14. The system according to claim 12, wherein the rotating mixer is oval shaped, which creates a shearing effect to mix the powder and liquid components to a homogenous bone fill material mix.

15. The system according to claim 12, further comprises an extractor, used for manual transfer of bone fill material from the mixer to the injector; wherein the extractor comprises a transfer piston, which is assembled to the cement mixing reservoir at distal end and to the force applicator of the injector at proximal end.

16. The system according to claim 15, wherein a pressure on the upper edge of the transfer piston causes the piston to descend into the mixing reservoir, thereby displacing the cement upwards into the main body of the force applicator.

17. A method for injecting bone fill material into an implant area comprising: mixing a powder component and a liquid component of bone fill material together in a mixer, using an extractor to transfer the mixed bone fill material from the mixer to an remotely controlled motor powered injector; and injecting the mixed bone fill material into the implant area, wherein the mixer is oval shaped and wherein the motor is a reduction motor.

18. The method according to claim 17, wherein the injector comprises:
(i) a force applicator component, with a proximal end and a distal end, comprising:
a main body, a piston slidably disposed within the main body, and a motor; and wherein the force applicator component is controlled by a remote control device
(ii) a delivery reservoir component comprising a reservoir and an outlet, wherein the delivery reservoir component is releasably connected to the force applicator component.

19. The method according to claim 18, wherein the piston moves along a central axis within the main body of the injector.

20. The method according to claim 19, wherein movement of the piston is powered and/or controlled by the motor placed at or near the proximal end of the force applicator, wherein the motor is connected to the piston, and wherein a gasket connects the piston to delivery reservoir at the distal end of the force applicator.

21. The method according to claim 20, wherein the motor generates a force which is transferred to the piston for ejecting the bone fill material through the delivery outlet into an implant area.

22. The method according to claim 17, wherein the motor includes an electric motor.

23. The method according to claim 18, wherein the injector injects the bone fill material, and wherein at least one of the volume and flow rate of injected cement is accurately controlled via the motor.

24. The method according to claim 18, wherein the remote control device controls the motor via at least one of a wire and wireless connection.

25. The method according to claim 18, wherein the remote control comprises one or more of the following:
a) a first screen for showing the time spent and remaining for injecting bone fill material;
b) a second screen for showing the cumulative volume of the injected bone fill material;
c) a button for injecting low volume of bone fill material;
d) a button for injecting moderate volume of bone fill material;
e) a button for injecting high volume of bone fill material;
f) a button of departure countdown;
g) a switch button to turn on or off the remote control;
h) a light to indicate that transfer has begun;
i) a light to indicate that injection has begun;
j) a light to indicate that injection has ended; and
k) a button to stop injection in an emergency.

26. The method according to claim 18, wherein the motor is connected to the piston with circlips.

27. The method according to claim 18, wherein the delivery outlet is in fluid communication with a cannula or a needle.

28. The method according to claim 18, wherein the shape of the force applicator component is cylindrical.

29. The method according to claim 17, wherein the mixer comprises two or more of the following:
(i) a mixing reservoir;
(ii) a rotating mixer;
(iii) a crank; and
(iv) a cover;
wherein the crank is connected at one end to the handle and at the other end to the rotating mixer, and the rotating mixer is rotated with the crank for mixing a powder component and a liquid component of the bone fill material in the mixing reservoir.

30. The method according to claim 29, wherein the mixer further comprises a scraper, wherein the scraper moves along the body of the mixer by rotating along matched threads within the reservoir and is separable from the reservoir by rotating in the reverse direction, and holds down the bone fill material in the reservoir once the cover is removed.

31. The method according to claim 30, wherein the rotating mixer creates a shearing effect to mix the powder and liquid components to a homogenous bone fill material mix.

32. The method according to claim 17, wherein the extractor is used for manual transfer of bone fill material from the mixer to the injector; wherein the extractor comprises a transfer piston, which is assembled to the cement mixing reservoir at distal end and to the force applicator of the injector at proximal end.

33. The method according to claim 32, wherein a pressure on the upper edge of the transfer piston causes the piston to descend into the mixing reservoir, thereby displacing the cement upwards into the main body of the force applicator.
